(19) United States
(12) Patent Application Publication
(10) Pub. No.: US 2015/0018816 A1
(43) Pub. Date: Jan. 15, 2015

(54) ELECTRODE ASSEMBLY FOR USE WITH SURGICAL INSTRUMENTS

(71) Applicant: COVIDIEN LP, Mansfield, MA (US)

(72) Inventor: CASSANDRA LATIMER, THORNTON, CO (US)

(73) Assignee: COVIDIEN LP, Mansfield, MA (US)

(21) Appl. No.: 14/182,967

(22) Filed: Feb. 18, 2014

Related U.S. Application Data
(60) Provisional application No. 61/845,226, filed on Jul. 11, 2013.

(51) Int. Cl. A61B 18/14

(52) U.S. Cl. CPC A61B 18/1445
USPC 606/41

(57) ABSTRACT

A system for treating tissue is provided. The system includes a source of electrosurgical energy and one or more microcontrollers. A surgical instrument is electrically coupled to the source of electrosurgical energy and includes an end effector including a pair of first and second jaw members. An electrode assembly includes first and second electrodes that are configured to selectively couple to the first and second jaw members, respectively, for treating. One or both of the first and second electrodes includes one or more pressure sensors configured to communicate a pressure between the first and second jaw members to the microcontroller as the first and second jaw members grasp tissue. One or both of the first and second electrodes includes one or more position sensors configured to provide position information to the microcontroller as the first and second electrodes grasp tissue.

### CROSS REFERENCE TO RELATED APPLICATION

The present application claims the benefit of and priority to U.S. Provisional Application Ser. No. 61/845,226, filed on Jul. 11, 2013, the entire contents of which are incorporated herein by reference.

### BACKGROUND

1. Technical Field

The present disclosure relates to an electrode assembly for use with surgical instruments. More particularly, the present disclosure relates to an electrode assembly that is configured to selectively couple to an end effector of a surgical instrument for treating tissue.

2. Description of Related Art

Endoscopic surgeries such as laparoscopic and thoracoscopic procedures are a less invasive alternative to traditional open surgeries. For example, laparoscopic procedures typically involve one or more relatively small (e.g. about 5 to 12 mm) incisions in a patient's abdominal area to provide entry for various instruments including cutting, grasping and positioning instruments as well as viewing devices to enable the physician to perform the surgery.

A number of grasping devices have been employed for performing such surgical procedures, e.g., to hold and move one or more of a patient's organs or other tissue so the physician can carry out the desired surgery. Typically, such grasping devices are not configured to transmit electrosurgical energy to tissue for treatment thereof.

### SUMMARY

As can be appreciated, an electrode assembly that is configured to selectively couple to an end effector of a surgical instrument for treating tissue may prove useful in the surgical arena.

Embodiments of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

An aspect of the present disclosure provides a system for treating tissue. The system includes a source of electrosurgical energy. One or more microcontrollers are configured to operably communicate with the source of electrosurgical energy. A surgical instrument is electrically coupled to the source of electrosurgical energy and includes an end effector including a pair of first and second jaw members for treating tissue. The first and second jaw members are movable from an open configuration to a clamping configuration for grasping tissue therebetween. An electrode assembly includes first and second electrodes that are configured to selectively couple to the first and second jaw members, respectively. One or both of the first and second electrodes includes one or more pressure sensors that are configured to communicate a pressure between the first and second jaw members to the microcontroller as the first and second jaw members grasp tissue. One or both of the first and second electrodes includes one or more position sensors that are configured to provide position information to the microcontroller as the first and second electrodes grasp tissue.

The first and second electrodes each couple to a respective lead that is provided on the surgical instrument. The leads are in electrical communication with the source of electrosurgical energy for providing electrosurgical energy to the first and second electrodes. The microcontroller may be a component of the source of electrosurgical energy. The source of electrosurgical energy may be configured to generate RF energy to the first and second electrodes. The first and second electrodes may couple to the respective first and second jaw members by way of a magnetic interface.

An aspect of the present disclosure provides a surgical instrument adapted to electrically couple to a source of electrosurgical energy in operable communication with one or more microcontrollers. An end effector includes a pair of first and second jaw members that are movable from an open configuration to a clamping configuration for grasping tissue therebetween. An electrode assembly including first and second electrodes is configured to selectively couple to the first and second jaw members, respectively. One or both of the first and second electrodes is/are activatable for treating tissue. One or both of the first and second electrodes includes one or more pressure sensors that are configured to communicate a pressure between the first and second jaw members to the microcontroller as the first and second jaw members grasp tissue. One or both of the first and second electrodes includes one or more position sensors that are configured to provide position information to the microcontroller as the first and second electrodes grasp tissue.

The first and second electrodes each couple to a respective lead that is provided on the surgical instrument. The leads may be in electrical communication with the source of electrosurgical energy for providing electrosurgical energy to the first and second electrodes. The first and second electrodes may couple to the respective first and second jaw members by way of a magnetic coupling configuration and/or a clip.

An aspect of the present disclosure provides a method for electrosurgically treating tissue. First and second electrodes of an electrode assembly are coupled to first and second jaw members of a surgical instrument. Tissue of interest is grasped. A position of the first and second electrodes relative to one another along one or more axes is measured and position information is communicated to a microcontroller as the jaw members grasp tissue. A pressure between the first and second jaw members as the first and second jaw members are grasping tissue is measured and pressure information is communicated to the microcontroller. Electrosurgical energy is transmitted from an electrosurgical energy source to the first and second electrodes for treating the tissue based on the position and pressure information provided by the microcontroller.

Upon completion of electrosurgically treating tissue, a first end tone from the source of electrosurgical energy may be generated if the position and the pressure information satisfy a first condition and a second end tone from the source of electrosurgical energy may be generated if one of the position and pressure information fails to satisfy the first condition.

The first and second electrodes may be coupled to respective leads provided on the surgical instrument. The leads may be in electrical communication with the source of electrosurgical energy for providing electrosurgical energy to the first and second electrodes. The microcontroller may be provided as a component of the source of electrosurgical energy. RF energy may be generated to the first and second electrodes. A magnetic coupling interface may be utilized for coupling the first and second electrodes to the respective first and second jaw members.

### BRIEF DESCRIPTION OF THE DRAWING

Various embodiments of the present disclosure are described hereinbelow with references to the drawings, wherein:

FIG. 1 is a perspective view of a surgical instrument that is configured for use with an electrode assembly in accordance with an embodiment of the instant disclosure;

FIG. 2 is a plan view of outside surfaces of an electrode assembly including first and second electrodes configured for use with the surgical instrument shown in FIG. 1;

FIG. 3 is a plan view of inside surfaces of the first and second electrodes shown in FIG. 2; and

FIG. 4 is a partial, perspective view of the jaw members of the surgical instrument shown in FIG. 1 with the electrode assembly shown in FIGS. 2 and 3 separated relative thereto.

### DETAILED DESCRIPTION

Detailed embodiments of the present disclosure are disclosed herein; however, the disclosed embodiments are merely examples of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

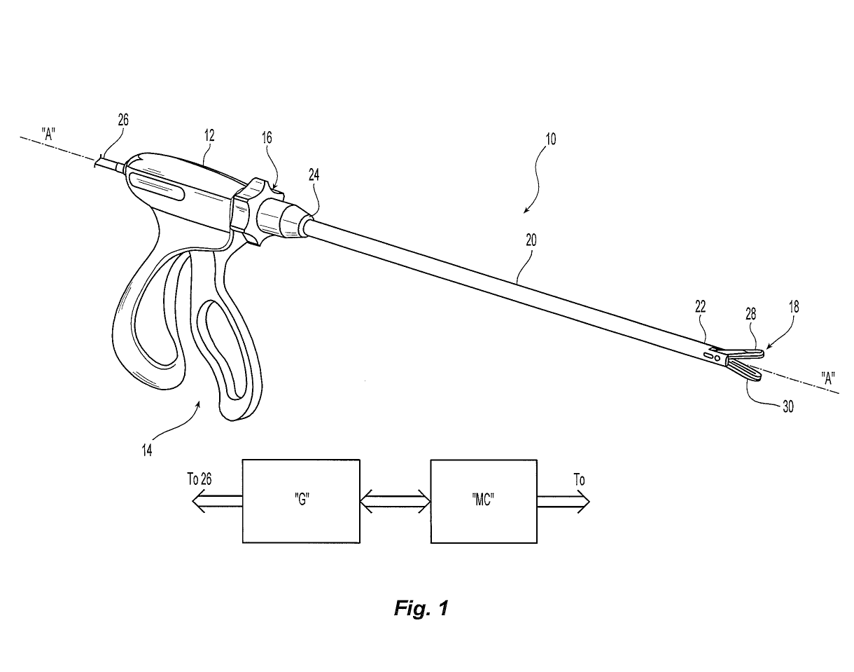

FIG. 1 illustrates a surgical instrument 10 (e.g., a grasper 10). Briefly, surgical instrument 10 includes a housing 12, a handle assembly 14, a rotating assembly 16 and an end effector assembly 18. A shaft 20 extends from the housing 12 and has a longitudinal axis “A-A” defined therethrough. A distal end 22 of shaft 20 is configured to mechanically engage end effector assembly 18 and a proximal end 24 is configured to mechanically engage housing 12. Surgical instrument 10 includes an electrosurgical cable 26 that connects to a generator “G” or other suitable power source (FIG. 1). The surgical instrument 10 may alternatively be configured as a battery-powered instrument. The generator “G” (e.g., Force Triad manufactured by Covidien) may be configured to provide electrosurgical energy (e.g., RF, microwave, optical, etc.,), thermal energy, ultrasonic energy, and the like to jaw members 28 and 30 of the end effector assembly 18. A microcontroller “MC” is configured to control operation of the generator “G.” The jaw members 28, 30 are pivotally mounted for movement between an open configuration for positioning tissue therebetween to a clamping configuration for grasping the tissue. Although in the illustrated embodiment both jaw members 28, 30 move between open and closed positions, it is also contemplated that one of the jaw members could be stationary and the other jaw member movable between open and closed positions. The jaw members 28, 30 can have teeth about their respective peripheries to enhance their grasping function.

For a more detailed description of the surgical instrument 10, reference is made to commonly-owned U.S. Patent Publication No. 2011/0230910 filed by Stopek on Feb. 16, 2011.

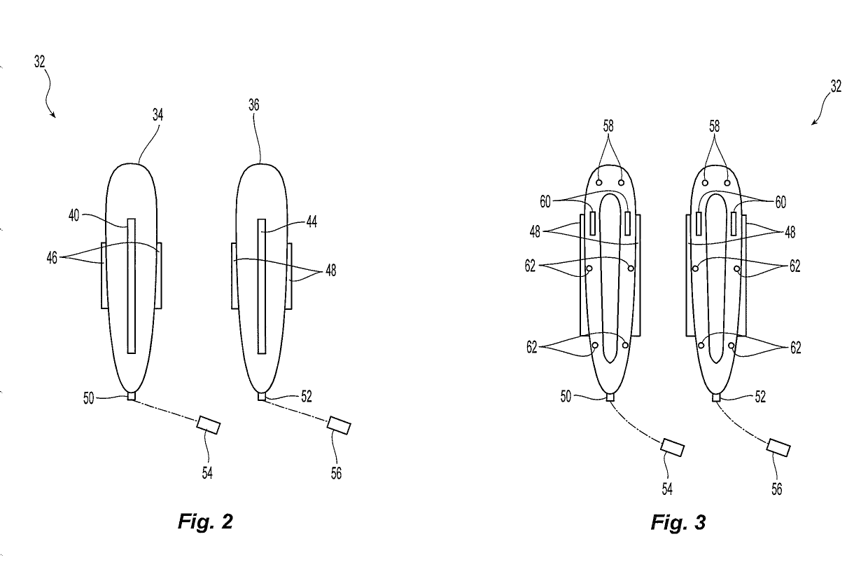

Referring to FIGS. 2-4, an electrode assembly 32 configured to selectively couple to jaw members 28, 30 of the end effector 18. In accordance with the instant disclosure, electrode assembly 32 may provide surgeons with the ability to choose a specific electrode assembly 18 for a specific jaw configuration and shaft length. Electrode assembly 32 includes first and second electrodes 34, 36 having a shape that complements a shape of the jaw members 28, 30.

The electrodes 34, 36 are configured to selectively couple to respective first and second jaw members 28, 30 via one or more suitable coupling methods. In some embodiments, for example, a magnetic coupling may be utilized to allow a user to selectively couple the electrodes 34, 36 to the respective jaw members 28, 30. In this particular embodiment, a magnetic interface 38, e.g., a magnetic (or ferromagnetic) strip, may be provided on an interior surface of each jaw member, e.g., jaw member 28 (FIG. 4) and a corresponding magnetic interface 40, e.g., a magnetic (or ferromagnetic) strip, may be provided on an exterior surface of each electrode, e.g., electrode 34 (FIG. 2). Similarly, a magnetic interface 42, e.g., a magnetic (or ferromagnetic) strip, may be provided on an interior surface of the other jaw member 30 (FIG. 1) and a corresponding magnetic interface 44, e.g., a magnetic (or ferromagnetic) strip, may be provided on an exterior surface of the other electrode 36 (FIG. 2).

Alternatively, electrodes 34, 36 may include lateral extensions 46, 48 that are provided on opposing side surfaces of the respective electrodes 34, 36. Lateral extensions 46, 48 function as clips and allow a user to selectively snap the electrodes 34, 36 on to or into the respective jaw members 28, 30. Lateral extensions 46, 48 may be resilient to facilitate coupling and uncoupling the electrodes 34, 36 to and from the jaw members 28, 30. As can be appreciated, other coupling methods and/or processes may be utilized to couple the electrodes 34, 36 to the respective jaw members 28, 30.

The electrodes 34, 36 include electrical contacts 50, 52 that engage a respective lead 54, 56 provided on the jaw members 28, 30, respectively, when the electrodes 34, 36 are coupled to the jaw members 34, 36. The leads 54, 56 are in electrical communication with the generator “G” via the electrosurgical cable 26 for providing electrosurgical energy to the electrodes 34, 36. The electrical contacts 50, 52 are in electrical communication (e.g., via wires, electrical traces, etc.) with one or more sensors 58, 60 that are provided on one or both of the jaw members 28, 30. Engagement between electrical contacts 50, 52 and leads 54, 56 provides a data transmission path to the microcontroller “MC” for data collected by the sensors 58, 60.

The electrodes 34, 36 may be configured for operation with various surgical procedures. In the illustrated embodiment, for example, the electrodes 34, 36 may be configured for use in a tissue sealing procedure. In this embodiment, sensors 58, 60 assist with tissue seal reliability and are provided on the electrodes 34, 36 to determine whether the electrodes 34, 36 are properly aligned/positioned on the jaw members 28, 30 and whether an adequate amount of pressure is being applied on tissue grasped between the electrodes 34, 36 so that a quality tissue seal may be achieved at the tissue.

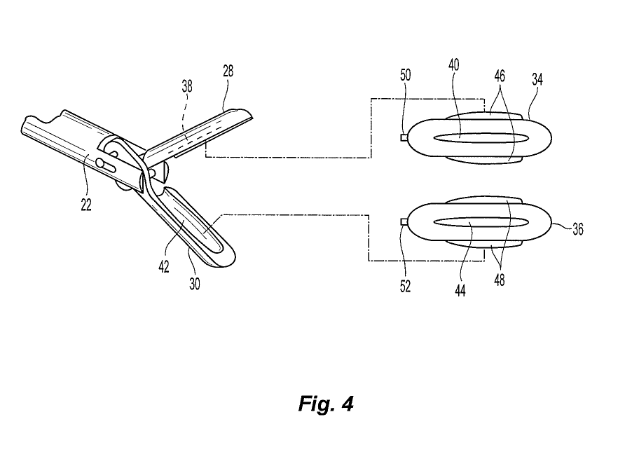

In some embodiments, the sensors 60 are configured to measure pressure between the jaw members 28, 30 when the electrodes 34, 36 are coupled to the jaw members 28, 30 and the jaw members 28, 30 are in the closed configuration with tissue grasped therebetween. It has been found that pressure between the jaw members 28, 30 ranging from about 3 kg/cm³ to about 16 kg/cm³ is adequate for sealing tissue. Moreover, the sensors 58 (e.g., proximity sensors, accelerometers, etc.) are configured to measure an alignment/position of the electrodes 34, 36 relative to one another along one or more axes to ensure that the electrodes 34, 36 are properly aligned with one another and to provide a specific gap distance therebetween. In some embodiments, the sensors 58 may be configured to cooperate with one or more adjustable or flexible stop members 62 (FIG. 3) to provide a gap distance between the electrodes 34, 36 that ranges from about 0.001 inches to about 0.006 inches. In some embodiments, one or both of the electrodes 34, 36 may include one or more non-conductive stop members 62 to ensure that the above referenced specific gap distances are maintained between the electrodes 34, 36. For a more detailed description of stop members 62, reference is made to U.S. Pat. No. 7,473,253 to Dycus et al. and U.S. Pat. No. 7,491,201 to Shields et al. The sensors 58, 60 may be operable before, during and/or after the tissue sealing procedure for communicating the aforementioned alignment/position and pressure information to the microcontroller “MC.” Further, the microcontroller “MC” (or control algorithms associated therewith) may be configured to control the amount of electrosurgical energy based on the information provided to the sensors 58, 60. All of these three factors may contribute in providing an effective, uniform and consistent tissue seal.

In use, electrode assembly 32 may be shipped and packaged in a sterile environment. A user may choose a surgical instrument, e.g., a grasper 10, suitable for use in performing a specific surgical procedure (e.g., a tissue sealing procedure). The surgeon may couple the electrodes 34, 36 to the jaw members 28, 30, respectively. When the electrodes 34, 36 are coupled to the jaw members 28, 30, contacts 50, 52 of electrodes 34, 36, respectively, engage leads 54, 56 provided on jaw members 28, 30, respectively.

In some embodiments, sensors 58 may be utilized to determine if the electrodes 34, 36 are properly aligned with one another. Specifically, sensors 58 communicate alignment data to the microcontroller “MC” which then records this information in memory associated with the microcontroller “MC” and/or generator “G.”. Thereafter, tissue may be positioned between the electrodes 34, 36 and grasped therebetween. Sensors 60 communicate the pressure data to the microcontroller “MC” which then records this information in memory. Moreover, the sensors 58 communicate the gap distance between the electrodes 34, 36. If the microcontroller “MC” determines that the electrodes 34, 36 meet the above mentioned pressure, gap and alignment requirements (e.g., a first condition), electrosurgical energy (or the required amount and intensity of electrosurgical energy) is transmitted to the electrodes 34, 36 to seal tissue. Moreover, the generator “G” and/or microcontroller “MC” may be configured to generate a first end tone when the tissue sealing procedure is completed and if the first condition is satisfied.

If the microcontroller “MC” determines that the electrodes 34, 36 do not meet one or more of the above mentioned pressure, gap and/or alignment requirements, a different amount or intensity of electrosurgical energy may be transmitted to the electrodes 34, 36 to electrosurgically treat (e.g., coagulate tissue) tissue or the user will be alerted to an error. In this instance, however, the generator “G” and/or microcontroller “MC” may be configured to generate a second end tone that is different from the first end tone to alert the user.

The electrode assembly 32 provides a surgeon with the capability of being able to choose the best set of jaw electrodes 34, 36 for the specific procedure. One or more electrodes 34, 36 or sets of electrodes 34, 36 with different parameters may be quickly interchanged before/during the electrosurgical procedure. For example, electrodes with varying thickness or thermal conductivity may be utilized depending upon a tissue type. Further, electrodes with differently-sized stop members may be utilized depending on tissue type. Moreover, the electrode assembly 32 provides a surgeon with the capability of being able to use one or more compatible surgical graspers, e.g., grasper 10, that best fit the surgeon's ergonomic needs while having the ability to electrosurgically treat tissue, e.g., seal tissue, coagulate tissue, and/or desiccate tissue.

From the foregoing and with reference to the various figure drawings, those skilled in the art will appreciate that certain modifications can also be made to the present disclosure without departing from the scope of the same. For example, the various embodiments disclosed herein may also be configured to work with robotic surgical systems and what is commonly referred to as “Telesurgery”. Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. These sensors may be configured to cooperate with one or more of the above sensors 58 and sensors 60. The master handles may also include a variety of different actuators for delicate tissue manipulation or treatment further enhancing the surgeon's ability to mimic actual operating conditions.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A system for treating tissue, comprising:
a source of electrosurgical energy;
at least one microcontroller in operable communication with the source of electrosurgical energy;
a surgical instrument electrically coupled to the source of electrosurgical energy and including an end effector including a pair of first and second jaw members movable from an open configuration to a clamping configuration for grasping tissue; and
an electrode assembly including first and second electrodes configured to selectively couple to the first and second jaw members, respectively, for treating tissue, at least one of the first and second electrodes including at least one pressure sensor configured to communicate a pressure between the first and second jaw members to the at least one microcontroller as the first and second jaw members grasp tissue, at least one of the first and second electrodes including at least one position sensor configured to provide position information to the microcontroller as the first and second electrodes grasp tissue.

2. The system according to claim 1, wherein the first and second electrodes each couple to a respective lead provided on the surgical instrument, the leads in electrical communication with the source of electrosurgical energy for providing electrosurgical energy to the first and second electrodes.

3. The system according to claim 1, wherein the microcontroller is a component of the source of electrosurgical energy.

4. The system according to claim 1, wherein source of electrosurgical energy is configured to generate RF energy to the first and second electrodes.

5. The system according to claim 1, wherein the first and second electrodes couple to the respective first and second jaw members by way of a magnetic interface.

6. A surgical instrument adapted to electrically couple to a source of electrosurgical energy in operable communication with at least one microcontroller, comprising:
an end effector including a pair of first and second jaw members movable from an open configuration to a clamping configuration for grasping tissue; and
an electrode assembly including first and second electrodes configured to selectively couple to the first and second jaw members, respectively, at least one of the first and second electrodes activatable for treating tissue, at least one of the first and second electrodes including at least one pressure sensor configured to communicate a pressure between the first and second jaw members to the microcontroller as the first and second jaw members grasp tissue, at least one of the first and second electrodes including at least one position sensor configured to provide position information to the microcontroller as the first and second electrodes grasp tissue.

7. The surgical instrument according to claim 6, wherein the first and second electrodes each couple to a respective lead provided on the surgical instrument, the leads in electrical communication with the source of electrosurgical energy for providing electrosurgical energy to the first and second electrodes.

8. The surgical instrument according to claim 6, wherein the first and second electrodes couple to the respective first and second jaw members by way of a magnetic interface.

9. A method for electrosurgically treating tissue, comprising:
coupling first and second electrodes of an electrode assembly to first and second jaw members of a surgical instrument;
grasping tissue of interest;
measuring a position of the first and second electrodes relative to one another along one or more axes;
communicating position information of the first and second electrodes to a microcontroller as the first and second jaw members grasp tissue;
measuring a pressure between the first and second jaw members when the first and second jaw members are grasping tissue;
communicating pressure information to the microcontroller; and
transmitting electrosurgical energy from an electrosurgical energy source to the first and second electrodes for treating the tissue based on the position and pressure information provided by the microcontroller.

10. The method according to claim 9, further including:
generating a first end tone from the source of electrosurgical energy if the position and pressure information satisfy a first condition; and
generating a second end tone from the source of electrosurgical energy if one of the position and pressure information fails to satisfy the first condition.

11. The method according to claim 10, further including coupling the first and second electrodes to respective leads provided on the surgical instrument, the leads in electrical communication with the source of electrosurgical energy for providing electrosurgical energy to the first and second electrodes.

12. The method according to claim 10, further including providing the microcontroller as a component of the source of electrosurgical energy.

13. The method according to claim 9, further including generating RF energy to the first and second electrodes.

14. The method according to claim 9, further including utilizing a magnetic coupling interface to couple the first and second electrodes to the respective first and second jaw members.
